(19) United States
(12) Patent Application Publication
(10) Pub. No.: US 2014/0275562 A1
(43) Pub. Date: Sep. 18, 2014

(54) PREPARATION OF CERTAIN (SUBSTITUTED PHENYL)-TRIAZOLYL-(SUBSTITUTED PHENYL) MOLECULES, AND INTERMEDIATES AND INSECTICIDES RELATED THERETO

(71) Applicant: Dow AgroSciences LLC, Indianapolis, IN (US)

(72) Inventors: James M. RENGA, Indianapolis, IN (US); Anne M. WILSON, Indianapolis, IN (US); Peter BORROMEO, Fishers, IN (US); Carl DeAMICIS, Indianapolis, IN (US); Jerod PATZNER, Indianapolis, IN (US)

(73) Assignee: Dow AgroSciences LLC, Indianapolis, IN (US)

(21) Appl. No.: 14/192,433

(22) Filed: Feb. 27, 2014

Related U.S. Application Data
(60) Provisional application No. 61/778,523, filed on Mar. 13, 2013.

(51) Int. Cl. C07D 249/08

(52) U.S. Cl. CPC C07D 249/08
USPC 548/269.2

(57) ABSTRACT

This disclosure is related to the field of preparation of certain (substituted phenyl)-triazolyl-(substituted phenyl) molecules, and intermediates related thereto, where said intermediates are useful in the preparation of certain insecticides.

### CROSS-REFERENCE TO RELATED APPLICATIONS

This application claims the benefit of U.S. Provisional Patent Application Ser. No. 61/778,523 filed Mar. 13, 2013, the entire disclosure of which is hereby expressly incorporated by reference.

### FIELD OF THE DISCLOSURE

This disclosure is related to the field of preparation of certain (substituted phenyl)-triazolyl-(substituted phenyl) molecules, and intermediates related thereto, where said intermediates are useful in the preparation of certain insecticides.

### BACKGROUND OF THE DISCLOSURE

U.S. Pat. No. 8,178,658 discloses pesticidal compositions comprising a compound having the following structure:

[chemical structure]

wherein Ar₁, Het, Ar₂, J, L, K, Q, R1, R2, R3, and R4 are disclosed in the patent. While processes are disclosed on how to make such compounds, and such processes are useful, it is desired to have more useful processes to make these compounds. In particular, it is desirable to have more commercially useful routes (especially routes with fewer process steps) to certain substituted triaryl intermediates disclosed in the patent that are useful in producing the compounds of Formula A-1.

### DETAILED DESCRIPTION OF THE DISCLOSURE

Throughout this document, all temperatures are given in degrees Celsius, and all percentages are weight percentages of isolated products unless otherwise stated.

[chemical structure]

Arylalkoxyimidate salts of Formula 1.2, wherein R₁ is NO₂, C(═O)OH or a (C₁-C₆) ester thereof (C(═O)O(C₁-C₆)alkyl), for example, methyl (C(═O)OCH₃) or ethyl ester (C(═O)OCH₂CH₃), can be prepared as outlined in Scheme 1. In step a, benzonitriles of Formula 1.1 are reacted with anhydrous inorganic acids in an alcohol to produce said arylalkoxyimidate salts, wherein R₂ is (C₁-C₆)alkyl.

In Step a benzonitriles of Formula 1.1 are treated with an anhydrous inorganic acid (HX, wherein X is F, Cl, Br, or I, preferably Cl or Br), for example, hydrogen chloride (HCl) or hydrogen bromide (HBr) in a polar protic solvent, for example, an alcohol (R₂OH), for example, methanol (MeOH), ethanol (EtOH), n-butanol, isopropanol, or mixtures thereof. In some embodiments, HX gas is introduced directly into a solution of the benzonitrile of Formula 1.1 in R₂OH via a sparge tube. The reaction is conducted at a temperature from about −10° C. to about −5° C. and preferably from about 0° C. to about −5° C. during the HX sparge. It is preferred if the temperature is raised to about 25° C. following the addition of the HX. HX gas may be introduced into the reaction system at pressures ranging from about atmospheric pressure to about 3500 kilopascals (kPa). Alternatively, solutions of benzonitriles of Formula 1.1, in a non-protic organic solvent such as, for example, tetrahydrofuran (THF), ethyl acetate (EtOAc), dichloromethane (CH₂Cl₂), benzene, toluene, xylenes, or mixtures thereof, are treated with an anhydrous inorganic acid (HX), for example, HCl or HBr, in the presence of an alcohol (R₂OH). Molar ratios of benzonitriles of Formula 1.1 to the alcohol are from about 1:1 to about 1:10, however, molar ratios of about 1:1000 to about 1000:1 may also be used. In another embodiment, HX is generated in situ via the decomposition of an acyl halide, such as, for example, acetyl chloride and acetyl bromide, when said acyl halide is contacted with R₂OH. In this method the acyl halide may be added to a solution of the benzonitrile of Formula 1.1 in R₂OH or may be added to the R₂OH first, followed by the addition of the benzonitrile of Formula 1.1 to the pre-formed solution of HX. In another embodiment, thionyl chloride is used as a source of HCl. In both cases, the reaction is conducted at a temperature from about −10° C. to about −5° C. and preferably from about 0° C. to about −5° C. during the HX formation, and preferably the temperature is raised to about 25° C. following the addition.

In some embodiments, subjecting benzonitriles of Formula 1.1, wherein R₁ is nitro or a benzoate ester, to one of the described methods affords alkoxyimidate salts of Formula 1.2, wherein R₁ is as defined and R₂ is derived from R₂OH. In another embodiment, subjecting benzonitriles of Formula 1.1, wherein R₁ is a carboxylic acid, to one of the described methods affords a mixture of alkoxyimidate salts of Formula 1.2, wherein R₁ is a carboxylic acid or an ester of a carboxylic acid, wherein the R₁ ester and R₂ are both derived from R₂OH, e.g., when R₂OH is MeOH, R₁ is the methyl ester and R₂ is a methyl.

[chemical structure]

Aryltriazoles of Formula 2.2, can be prepared as outlined in Scheme 2. In step a, arylalkoxyimidate salts of Formula 1.2, are reacted with formylhydrazine (H₂NNHCHO) to produce intermediate iminohydrazines of the Formula 2.1. In step b, the iminohydrazines are heated to facilitate cyclization to aryltriazoles of Formula 2.2.

In step a arylalkoxyimidate salts of Formula 1.2 are reacted with formylhydrazine. The reaction is carried out in a basic heterocyclic solvent, for example, pyridine, 2-methylpyridine, 3-methylpyridine, 4-methylpyridine, lutidines, or mixtures thereof. The reaction is conducted at a temperature from about −10° C. to about 5° C. and preferably from about −5° C. to about 0° C. during the addition of formylhydrazine to the arylalkoxyimidate salts, and then the temperature is preferably raised to about 25° C. following the addition. Approximately, a molar ratio of about 1:1.2 of the arylalkoxyimidate salts of Formula 1.2 to formyl hydrazine may be used, however, molar ratios of about 5:1 to about 1:5 may also be used. The reaction is conducted at about atmospheric pressure, however, higher or lower pressures can be used.

In step b iminohydrazines of Formula 2.1 are heated to facilitate cyclization to aryltriazoles of Formula 2.2. The reaction is carried out in an aprotic solvent, for example, benzene, toluene, xylenes, or mixtures thereof. The reaction is conducted at a temperature from about 100° C. to about 150° C. and preferably from about 120° C. to about 140° C. The reaction is conducted at about atmospheric pressure, however, higher or lower pressures can be used.

[chemical structure]

1,3-Diaryltriazoles of Formula 3.2, wherein R is (C₁-C₆)haloalkoxy preferably trifluoromethoxy or pentafluoroethoxy, can be prepared as outlined in Scheme 3. In step a, aryltriazoles of Formula 2.2 are coupled with aryl halides of the Formula 3.1 (wherein Y is a F, Cl, Br, or I, preferably Br or I).

In step a aryltriazoles of Formula 2.2 are reacted with aryl halides of the Formula 3.1 in the presence of a metal catalyst, such as copper (I) iodide (CuI), copper (I) oxide (Cu₂O), or mixtures thereof, and a base for example, cesium carbonate (Cs₂CO₃), potassium phosphate (K₃PO₄), potassium carbonate (K₂CO₃), or mixtures thereof, with or without a ligand, for example, quinolin-8-ol or N,N′-dimethyl ethylenediamine or other 1,2-diamines or glycine, in a polar aprotic solvent such as acetonitrile (MeCN), dimethylformamide (DMF), dimethyl sulfoxide (DMSO), N-methylpyrrolidone (NMP), or mixtures thereof. The reaction is conducted at a temperature from about 120° C. to about 160° C. and preferably from about 140° C. to about 150° C. About a 1:1 to about 1:2 molar ratio of the aryltriazoles of Formula 2.2 to aryl halides of Formula 3.1 may be used, however, molar ratios of about 5:1 to about 1:5 may also be used. The reaction is conducted at about atmospheric pressure, however, higher or lower pressures can be used.

[chemical structure]

1,3-Diaryl triazole compounds of Formula 4.2 and Formula 4.3, can be prepared according to Scheme 4. In method a, intermediate 1,3-diaryltriazoles of Formula 3.2, wherein R₁ is an ester, can be saponified to give 1,3-diaryltriazoles substituted with a carboxylic acid of Formula 4.2. In method b, intermediate 1,3-diaryltriazole of Formula 3.2, wherein R₁ is nitro, can be reduced to give 1,3-diaryltriazoles substituted with an amine of Formula 4.3.

Method a can be conducted in a polar protic solvent, such as an alcohol, for example, MeOH, EtOH, n-butanol, isopropanol, or mixtures thereof, or in a polar aprotic solvent such as THF, in the presence of an alkali hydroxide base, for example, sodium (NaOH), potassium (KOH), or lithium hydroxide (LiOH), and water. The reaction can be conducted at a temperature from about 20° C. to about 60° C. and preferably from about 20° C. to about 30° C. The pH of the reaction mixture is from about 8 to about 14 and preferably from about 10 to about 12.

Method b can be carried out in a wide variety of organic solvents including, for example, polar protic solvents, for example, MeOH, EtOH, n-butanol, isopropanol, or mixtures thereof, polar aprotic solvents, for example, THF and EtOAc, or organic acids, for example, acetic acid, in the presence of a catalyst, such as palladium on carbon, and a hydrogen source, for example hydrogen gas, ammonium salts, e.g., ammonium formate, and cyclohexadiene. The reaction can be conducted at a temperature from about 20° C. to about 50° C. and preferably from about 20° C. to about 30° C. The reaction can be conducted at a pressure from about 100 kPa to about 700 kPa and preferably from about 100 kPa to about 350 kPa.

1,3-Diaryltriazole of Formula 4.2 and Formula 4.3 can be used as intermediates to form pesticides as disclosed in U.S. Pat. No. 8,178,658.

### EXAMPLES

These examples are for illustration purposes and are not to be construed as limiting the disclosure to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Examples for which the temperature is described as “room temperature” were conducted in climate controlled laboratories with temperatures ranging from about 20° C. to about 24° C. Molecules are given their known names, named according to naming programs within ISIS Draw, Chem Draw or ACD Name Pro. If such programs are unable to name a molecule, the molecule is named using conventional naming rules. ¹H NMR spectral data are reported in ppm (δ) and were recorded at 300, 400 or 600 MHz, and ¹³C NMR spectral data are reported in ppm (δ) and were recorded at 75, 100 or 150 MHz, unless otherwise stated.

### Example 1

### Preparation methyl 4-(imino(methoxy)methyl)benzoate hydrochloride

[chemical structure]

To a magnetically stirred solution of methyl 4-cyanobenzoate (12.5 g, 78 mmol) in benzene (25 mL) and MeOH (7 mL) cooled to 0° C. was bubbled HCl for 3 hours (h). After storing in the refrigerator overnight, a heavy white material formed. The solid was filtered through a fritted glass funnel and washed with ether to furnish the title compound (17.5 g, 96%): mp 209-210° C.; ¹H NMR (400 MHz, CDCl₃) δ 13.05 (br s, 1H), 12.32 (br s, 1H), 8.48 (m, 2H), 8.22 (m, 2H), 4.60 (s, 3H), 3.97 (s, 3H); ¹³C NMR (101 MHz, DMSO-d₆) δ 166.98, 165.69, 138.38, 131.74, 129.00, 127.78, 52.29, 26.16; EIMS m/z 192 ([M+]).

### Example 2

### Preparation of methyl 4-(ethoxy(imino)methyl)benzoate hydrochloride

[chemical structure]

A 2 L, three-necked round bottomed flask equipped with a magnetic stir bar, a temperature probe, addition funnel and nitrogen inlet was charged with methyl 4-cyanobenzoate (100 g, 620 mmol). The methyl 4-cyanobenzoate was taken up in EtOH (438 mL) and cooled in an ice bath to 0° C. Acetyl chloride (353 mL, 4960 mmol) was added dropwise into the stirring solution over a 2 h period during which time the reaction exothermed from 0° C. to 21° C. The reaction flask was stoppered and sealed with Parafilm® and allowed to stir at 23° C. for 18 h. A white precipitate started to crash out during the overnight stirring period. The solid was collected by vacuum filtration and washed with EtOH. The filtrate was then concentrated until turning cloudy. The mixture was cooled to 23° C. to allow for the imidate salt to crash out of solution. The first crop was collected via vacuum filtration and rinsed with EtOH. A second crop was also collected. The combined solids were dried to constant mass which furnished the title compound as a white solid (128 g, 85%): ¹H NMR (400 MHz, CDCl₃) δ 12.85 (br s, 1H), 12.20 (br s, 1H), 8.49 (m, 2H), 8.23 (m, 2H), 5.00 (q, 2H), 4.00 (s, 3H), 1.72 (t, 3H).

### Example 3

### Preparation of 4-(ethoxy(imino)methyl)benzoic acid, hydrochloride and ethyl 4-(ethoxy(imino)methyl)benzoate, hydrochloride

[chemical structure]

A 500 mL, three-necked flask, fitted with magnetic stirring, nitrogen outlet, addition funnel and a temperature probe was charged with dry EtOH (125 mL). The vessel was cooled down to 5° C. and acetyl chloride (97 mL, 1332 mmol) was slowly added while maintaining the temperature between 5° C. and 10° C. When the addition was complete, 4-cyanobenzoic acid (25 g, 167 mmol) was added in portions as a solid over 15 minutes (min). No exotherm was noted during the addition of the solid. When the addition was complete, the white suspension was allowed to warm to 25° C. The reaction vessel was then sealed with Parafilm® and allowed to stir at 23° C. for 18 h. The white suspension was vacuum filtered and the solid rinsed with EtOH and dried to constant mass, furnishing 4-(ethoxy(imino)methyl)benzoic acid hydrochloride as a white solid (25 g, 65%): ¹H NMR (400 MHz, DMSO-d₆) δ 12.44 (br s, 1H), 8.26 (m, 2H), 8.14 (m, 2H), 4.70 (q, 2H), 1.51 (t, 3H).

The filtrate was concentrated and treated with ether to give a white solid. The solid was collected by vacuum filtration and rinsed with ether to give ethyl 4-(ethoxy(imino)methyl)benzoate hydrochloride as a white solid (11 g, 25%): ¹H NMR (400 MHz, DMSO-d₆) δ 12.07 (br s, 1H), 8.24 (m, 2H), 8.15 (m, 2H), 4.66 (q, 3H), 4.37 (q, 3H), 1.49 (t, 3H), 1.35 (t, 3H).

### Example 4

### Preparation of ethyl 4-nitrobenzimidate hydrochloride

[chemical structure]

To 4-nitrobenzonitrile (27 g, 182 mmol) in EtOH (128 ml, 2187 mmol) cooled to 0° C. under a nitrogen atmosphere was added acetyl chloride (104 ml, 1458 mmol) dropwise over 1 h. The reaction was allowed to warm to room temperature. Once at room temperature, the reaction was stoppered and allowed to stir for 56 h. The resulting precipitate (4-nitrobenzamide) was collected on fritted funnel. Diethyl ether was added to the supernatant and the resulting precipitate was collected on a fritted funnel, washed with diethyl ether and air dried to give the title compound (26.7 g, 58%): mp 198-200° C.; ¹H NMR (400 MHz, DMSO-d₆) δ 8.37-8.30 (m, 2H), 8.21-8.13 (m, 2H), 7.35 (s, 1H), 7.22 (s, 1H), 7.09 (s, 1H), 4.34 (q, J=7.1 Hz, 2H), 1.32 (t, J=7.1 Hz, 3H); EIMS m/z 193 ([M+]).

### Example 5

### Preparation of methyl 4-(1H-1,2,4-triazol-3-yl)benzoate

[chemical structure]

To a magnetically stirred suspension of methyl 4-(imino(methoxy)methyl) benzoate hydrochloride (2.27 g, 11.75 mmol) in pyridine (10 mL) cooled to 0° C. was added formylhydrazine (0.776 g, 12.92 mmol) in three portions. The solution was allowed to warm to room temperature slowly over 1 h. Water (30 mL) was added and the suspension was filtered through a fritted funnel, washed with water and allowed to air dry to give methyl 4-((2-formylhydrazinyl)(imino)methyl)benzoate as a pale yellow solid (1.604 g, 66%): mp 139° C.; ¹H NMR (400 MHz, DMSO-d₆) δ (2:1 mixture of rotomers) major rotomer; 10.30 (brs, 1H), 8.04-7.93 (m, 2H), 7.93-7.84 (m, 2H), 6.56 (brs, 3H), 3.87 (s, 3H); ESIMS m/z 245 ([M+H+Na]⁺).

To a round bottom flask was suspended methyl 4-((2-formylhydrazinyl)(imino)methyl)benzoate (0.513 g, 2.32 mmol) in xylenes (8 mL). The pale yellow suspension was heated to 120° C. for 1.5 h. The yellow suspension slowly discolored and dissolved to a colorless solution. The reaction was allowed to cool to room temperature and a white solid precipitated from the solution. The white solid was filtered on a fritted glass funnel, washed with hexanes, air dried then dried under vacuum to furnish the title compound as a white solid (0.362 g, 77%): mp 194-195° C.; ¹H NMR (400 MHz, DMSO-d₆) δ 8.59 (s, 1H), 8.24-8.13 (m, 2H), 8.13-8.03 (m, 2H), 3.88 (s, 3H); ¹³C NMR (101 MHz, DMSO-d₆) δ 165.86, 134.68, 129.81, 129.72, 125.98, 119.83, 52.19; EIMS m/z 203 ([M+]).

### Example 6

### Preparation of 3-(4-nitrophenyl)-1H-1,2,4-triazole

[chemical structure]

Ethyl 4-nitrobenzimidate hydrochloride (3.03 g, 13.1 mmol) was placed in a round bottom flask with pyridine (11 mL) and cooled to 0° C. Formylhydrazine (0.947 g, 15.8 mmol) was added and the reaction was allowed to warm to room temperature slowly over 1 h. The yellow solution became a mustard yellow slurry. The reaction was diluted with water and the orange solid filtered on a fritted glass funnel, washed with water, air dried and then dried under vacuum to provide N′-formyl-4-nitrobenzimidohydrazide as a yellow-orange solid (1.84 g): mp 220-223° C.; ¹H NMR (400 MHz, DMSO-d₆) (3:1 mixture of rotomers) major rotomer: δ 10.39 (d, J=9.7 Hz, 1H), 8.31-8.23 (m, 2H), 8.09-7.95 (m, 2H), 6.66 (brs, 3H); ESIMS m/z 208 ([M+]).

N′-Formyl-4-nitrobenzimidohydrazide (2.362 g, 11.35 mmol) was placed in xylenes (45 mL) and heated to 140° C. for 4 h. The reaction was allowed to cool to room temperature with stirring overnight. The reaction was filtered through a fritted glass funnel and the pale yellow solid was washed with hexanes and air dried to furnish the title compound (1.436 g, 68%): mp 221-223° C.; ¹H NMR (400 MHz, DMSO-d₆) δ 14.40 (brs, 1H), 8.68 (brs, 1H), 8.34 (d, J=8.7 Hz, 2H), 8.27 (d, J=8.9 Hz, 2H); ESIMS m/z 190 ([M+]).

### Example 7

### Preparation of methyl 4-(1-(4-(trifluoromethoxy)phenyl)-1H-1,2,4-triazol-3-yl)benzoate and 4-(1-(4-(trifluoromethoxy)phenyl)-1H-1,2,4-triazol-3-yl)benzoic acid

[chemical structure]

To a nitrogen purged flask was added 1-iodo-4-(trifluoromethoxy)benzene (313 μl, 2.00 mmol), methyl 4-(1H-1,2,4-triazol-3-yl)benzoate (203 mg, 1 mmol), K₃PO₄ (531 mg, 2.50 mmol) that had been ground together with CuI (19.05 mg, 0.100 mmol) using a mortar and pestle and DMF (2 mL). The reaction was heated to 140° C. After 4 h, the reaction was allowed to coolto room temperature. The reaction was then diluted with water and EtOAc and the layers separated. The aqueous layer was extracted with EtOAc (3×10 mL) and the combined organic fractions were washed with water (10 mL) and brine (10 mL), dried over magnesium sulfate (MgSO₄), filtered and concentrated to give a yellow oil. The oil was taken up in EtOAc, washed with NaOH (1 N, 3×5 mL) and water (5 mL), dried over MgSO₄, filtered and concentrated. Purification by flash column chromatography (0-70% EtOAc/hexanes) provided methyl 4-(1-(4-(trifluoromethoxy)phenyl)-1H-1,2,4-triazol-3-yl)benzoate as a pale brown solid (25 mg, 7%): mp 166-168° C.; ¹H NMR (400 MHz, CDCl₃) δ 8.60 (s, 1H), 8.34-8.25 (m, 2H), 8.20-8.11 (m, 2H), 7.87-7.77 (m, 2H), 7.46-7.35 (m, 2H), 3.96 (s, 3H); ¹³C NMR (101 MHz, CDCl₃) δ 166.76, 162.63, 148.57, 141.78, 135.42, 134.46, 131.04, 130.02, 126.47, 122.45, 121.32, 52.24, 31.59; ESIMS m/z 363 ([M+]).

The combined aqueous layers were acidified to pH 2. The white solid was isolated on a fritted glass funnel to give 4-(1-(4-(trifluoromethoxy)phenyl)-1H-1,2,4-triazol-3-yl)benzoic acid as a tan solid (169 mg, 48%): mp 222-228° C.; ¹H NMR (400 MHz, DMSO-d₆) δ 13.01 (s, 1H), 9.47 (s, 1H), 8.30-8.19 (m, 2H), 8.15-8.03 (m, 4H), 7.64 (d, J=8.5 Hz, 2H); ESIMS m/z 349 ([M+]).

### Example 8

### Preparation of methyl 4-(1-(4-(perfluoroethoxy)phenyl)-1H-1,2,4-triazol-3-yl)benzoate and 4-(1-(4-(perfluoroethoxy)phenyl)-1H-1,2,4-triazol-3-yl)benzoic acid

[chemical structure]

Method 1 (using K₃PO₄). To a nitrogen purged round bottomed flask was added 1-bromo-4-(perfluoroethoxy)benzene (291 mg, 1 mmol), methyl 4-(1H-1,2,4-triazol-3-yl)benzoate (203 mg, 1.00 mmol), quinolin-8-ol (29.0 mg, 0.200 mmol), K₃PO₄ (531 mg, 2.50 mmol) that had been ground together with CuI (19.05 mg, 0.100 mmol) using a mortar and pestle and DMF (2 mL). The reaction was heated to 140° C. for 5.5 h, then allowed to cool to room temperature. The reaction was diluted with water and EtOAc. The aqueous layer was extracted with EtOAc (3×10 mL), and the combined organic fractions were washed with saturated aqueous sodium bicarbonate (NaHCO₃,10 mL), water (10 mL) and brine (10 mL), dried over MgSO₄, filtered and concentrated to give a brown oil. Purification by flash column chromatography (0-70% EtOAc/hexanes) provided methyl 4-(1-(4-(perfluoroethoxy)phenyl)-1H-1,2,4-triazol-3-yl)benzoate as a pale yellow solid (86 mg, 21%): mp 196-198° C.; ¹H NMR (400 MHz, CDCl₃) δ 8.61 (s, 1H), 8.32-8.25 (m, 2H), 8.19-8.11 (m, 2H), 7.88-7.77 (m, 2H), 7.47-7.37 (m, 2H), 3.96 (s, 3H); ¹³C NMR (101 MHz, CDCl₃) δ 166.76, 162.65, 147.75, 141.78, 135.59, 134.45, 131.05, 130.02, 126.47, 123.13, 121.29, 52.24; EIMS m/z 413 ([M+]).

The combined aqueous extracts were acidified to pH 2 and the resulting tan solid was collected on a fritted glass funnel to give 4-(1-(4-(perfluoroethoxy)phenyl)-1H-1,2,4-triazol-3-yl)benzoic acid (99.4 mg, 25%) as a light tan solid: mp 210-222° C.; ¹H NMR (400 MHz, DMSO-d₆) δ 13.00 (br s, 1H), 9.49 (d, J=3.4 Hz, 1H), 8.31-8.19 (m, 2H), 8.16-8.04 (m, 4H), 7.70-7.59 (m, 2H); ESIMS m/z 399 ([M+]).

Method 2 (using Cs₂CO₃). To a round bottom flask was added methyl 4-(1H-1,2,4-triazol-3-yl)benzoate (0.221 g, 1.088 mmol), 1-bromo-4-(perfluoroethoxy)benzene (0.317 g, 1.088 mmol), quinolin-8-ol (0.032 g, 0.218 mmol), CuI (0.021 g, 0.109 mmol) that had been ground together with Cs₂CO₃ (0.886 g, 2.72 mmol) using a mortar and pestle, DMF (2.2 mL) and water (0.22 mL). The reaction was heated to 140° C. for 16 h. After 2 h, a solid began to form and precipitate out on sides of flask. The solution was allowed to cool, then it was diluted with water and EtOAc. The layers were separated and the organic layer was extracted with water (1×20 mL), and saturated aqueous NaHCO₃ (2×20 mL). The aqueous layer was acidified and the resulting tan solid isolated on a fritted glass funnel (0.173 g). The supernatant was placed in the refrigerator for 14 h. The supernatant was filtered on a fritted glass funnel, washed with water and air dried to give 0.11 g of additional 4-(1-(4-(perfluoroethoxy)phenyl)-1H-1,2,4-triazol-3-yl)benzoic acid (0.283 g, 65%): mp 224-228° C.; ¹H NMR (400 MHz, DMSO-d₆) δ 13.00 (br s, 1H), 9.49 (d, J=3.4 Hz, 1H), 8.31-8.19 (m, 2H), 8.16-8.04 (m, 4H), 7.70-7.59 (m, 2H); ESIMS m/z 399 ([M+]).

### Example 9

### Preparation of 3-(4-nitrophenyl)-1-(4-(perfluoroethoxy)phenyl)-1H-1,2,4-triazole

[chemical structure]

To a nitrogen purged round bottomed flask was added 3-(4-nitrophenyl)-1H-1,2,4-triazole (0.24 g, 1.26 mmol), 1-bromo-4-(perfluoroethoxy)benzene (0.367 g, 1.26 mmol), quinolin-8-ol (0.048 g, 0.33 mmol), CuI (0.038 g, 0.20 mmol) that had been ground together with Cs₂CO₃ (0.822 g, 2.52 mmol) in a mortar and pestle, DMF (2.5 mL) and water (0.25 mL). The reaction was heated to 140° C. for 4 h, after which more 1-bromo-4-(perfluoroethoxy)benzene was added (few drops). After 4 h, TLC indicated that the triazole was consumed. The reaction was cooled and diluted with water and EtOAc. The layers were separated and the aqueous layer was extracted with EtOAc (3×20 mL). The organic fractions were washed with brine, dried over MgSO₄, filtered and concentrated to give a brown oil. Purification by flash column chromatography (0-70% EtOAc/hexanes) provided the title compound as a white solid (0.29 g, 57%): mp 133-135° C.; ¹H NMR (400 MHz, CDCl₃) δ 8.64 (s, 1H), 8.35 (m, 4H), 7.83 (m, 2H), 7.43 (m, 2H); ¹³C NMR (101 MHz, CDCl₃) δ 161.60, 148.53, 147.95, 142.08, 136.28, 135.40, 127.31, 124.06, 123.18, 121.36, 114.49, 96.41; EIMS m/z 400 ([M+]).

### Example 10

### Preparation of 4-(1-(4-(trifluoromethoxy)phenyl)-1H-1,2,4-triazol-3-yl)benzoic acid

[chemical structure]

To methyl 4-(1-(4-(trifluoromethoxy)phenyl)-1H-1,2,4-triazol-3-yl)benzoate (0.332 g, 0.914 mmol) in THF (6 mL) and water (3 mL) was added LiOH (0.066 g, 2.74 mmol). The solution immediately turned from yellow to orange-red. The reaction was stirred vigorously at room temperature for 16 h. The solution was acidified to pH 2 and diluted with water and CH₂Cl₂. The layers were separated and the aqueous layer was extracted with EtOAc (3×10 mL) and the combined organic fractions were washed with water (10 mL) and brine (10 mL), dried over MgSO₄, filtered and concentrated to give the title compound as a tan solid (0.29 g, 91%): mp 228-233° C.; ¹H NMR (400 MHz, DMSO-d₆) δ 10.55-10.24 (m, 1H), 9.46 (s, 1H), 8.23 (d, J=8.0 Hz, 2H), 8.09 (d, J=7.9 Hz, 4H), 7.64 (d, J=8.5 Hz, 2H); ESIMS m/z 350 ([M+H]⁺).

### Example 11

### Preparation of 4-(1-(4-(perfluoroethoxy)phenyl)-1H-1,2,4-triazol-3-yl)benzoic acid

[chemical structure]

In a 250 mL round bottomed flask equipped with an overhead stirrer, T-type thermocouple, and nitrogen inlet was added methyl 4-(1-(4-(perfluoroethoxy)phenyl)-1H-1,2,4-triazol-3-yl)benzoate (11.1 g, 26.9 mmol) and THF (100 mL). To this yellow suspension was added water (10 mL) and lithium hydroxide.monohydrate (3.4 g, 81 mmol). There was no change in reaction appearance and temperature (20.5° C.). The reaction was stirred at 23° C. for 39 h during which it became a yellow solution. The reaction was analyzed by LCMS which showed that the reaction had progressed only 23%. A heating mantle was attached to the reaction flask and the flask was heated to an internal temperature of 60° C. Analyse by LCMS showed the reaction to be complete. The reaction was cooled to 4° C. in an ice bath and water (100 mL) was added providing a light yellow solution. Concentrated HCl (8.0 g) was added (note: exothermic) which gave a thick white precipitate. The white suspension was stirred at 5° C. for 30 min then the solid was collected by vacuum filtration and washed with water (2×25 mL). The white wet cake was allowed to dry in air for 3 h, then placed into a vacuum oven (50° C., 700 mm Hg vacuum, 16 h). This gave the title compound as a white solid (10.3 g, 96%): mp 227-229° C.; ¹H NMR (400 MHz, CDCl₃) δ 8.65 (s, 1H), 8.32 (d, J=8.4 Hz, 2H), 8.23 (d, J=8.4 Hz, 2H), 7.84 (d, J=8.9 Hz, 1H), 7.42 (d, J=8.9 Hz, 2H).

What is claimed is:

1. A process comprising:
(1a) reacting an arylalkoxyimidate salt of Formula 1.2 with formylhydrazine to produce an iminohydrazine of Formula 2.1; followed by
(1b) cyclizing said iminohydrazine of Formula 2.1 to produce an aryltriazole of Formula 2.2; [chemical structure]
wherein
X is F, Cl, Br, or I;
R₁ is NO₂, C(═O)OH or a C(═O)O(C₁-C₆)alkyl;
R₂ is a (C₁-C₆) alkyl.

2. A process according to claim 1 further comprising reacting an aryltriazole of Formula 2.2 with an aryl halide of the Formula 3.1 to produce a 1,3-diaryltriazole of Formula 3.2 [chemical structure]
wherein
R is (C₁-C₆)haloalkoxy; and
Y is Br or I.

3. A process according to claim 1 wherein R₁ is C(═O)OCH₃ or C(═O)OCH₂CH₃.

4. A process according to claim 1 wherein the molar ratio of the arylalkoxyimidate salt of Formula 1.2 to the formylhydrazine is from about 5:1 to about 1:5.

5. A process according to claim 2 wherein R is trifluoromethoxy or pentafluoroethoxy.
